(19) United States
(12) Patent Application Publication
(10) Pub. No.: US 2020/0191762 A1
(43) Pub. Date: Jun. 18, 2020

(54) PROCEDURE FOR MEASURING TOTAL REACTIVE SULFUR

(71) Applicant: PHILLIPS 66 COMPANY, Houston, TX (US)

(72) Inventors: Ricky E. Snelling, Tulsa, OK (US); Omar J. Yepez, The Woodlands, TX (US); Madhu Anand, Bartlesville, OK (US); Donald R. Engelbert, Copan, OK (US); Jacqueline R. Webb, Bartlesville, OK (US); Heather M. Day, Bartlesville, OK (US)

(73) Assignee: PHILLIPS 66 COMPANY, HOUSTON, TX (US)

(21) Appl. No.: 16/712,205

(22) Filed: Dec. 12, 2019

Related U.S. Application Data
(60) Provisional application No. 62/779,072, filed on Dec. 13, 2018.

(51) Int. Cl. G01N 33/28; C10G 29/10

(52) U.S. Cl. CPC G01N 33/287; C10G 29/10

(57) ABSTRACT

Measuring the real corrosion risk that organosulfur compounds present in refinery operations is simplified by first measuring the total sulfur content of a sample of a hydrocarbon material. The sample is then combined with a specific quantity of high surface area iron powder at a temperature representative of the highest temperature anticipated in a refining process for a period of time, such as one hour. The solid phase is then removed, and the total sulfur content is again measured. The difference between the before and after represents the total reactive sulfur of the hydrocarbon material. The hydrocarbon material is then blended with other hydrocarbon materials to create a stream that can be optimized to utilize the maximum volume of the lowest cost feedstock while managing the corrosion risk to the refinery equipment and piping.

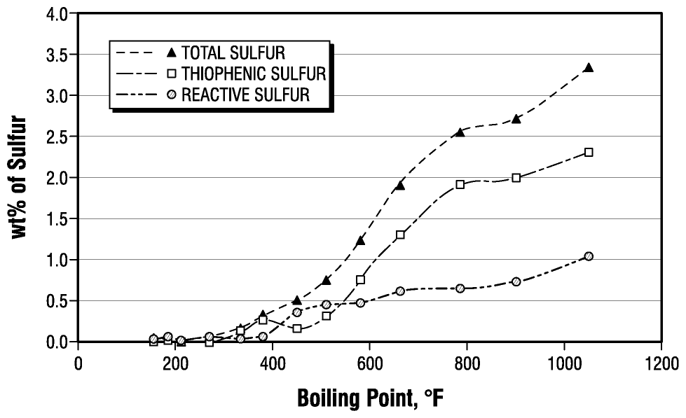

### CROSS-REFERENCE TO RELATED APPLICATIONS

This application is a non-provisional application which claims benefit under 35 USC § 119(e) to U.S. Provisional Application Ser. No. 62/779,072 filed Dec. 13, 2018, entitled “Procedure for Measuring Total Reactive Sulfur”, which is hereby incorporated herein in its entirety.

### STATEMENT REGARDING FEDERALLY SPONSORED RESEARCH OR DEVELOPMENT

None.

### FIELD OF THE INVENTION

This invention relates to refining hydrocarbons and particularly to blending sulfur containing hydrocarbon liquids for controlling corrosion in refinery systems and particularly to determining the corrosion risk of liquid hydrocarbons prior to submitting such liquids to refinery systems.

### BACKGROUND OF THE INVENTION

Crude oil refineries are continuously balancing the drive to efficiently refine large volumes of crude oil into refined products to make a profit while monitoring and reacting to numerous variables that limit productivity. One of the variables are corrosive components in crude oils available to refine and their relative concentration. Corrosiveness of refinery streams is a real problem in that concentrations of corrosive components in hydrocarbon streams can rapidly compromise equipment and piping in a refinery. Corrosive components are generally present in most areas of refineries and in numerous forms. One area of concern is in the heavier hydrocarbon streams from distillation processes where organosulfur compounds tend to concentrate. Crude oils with higher sulfur content are generally termed “sour crude” and, unfortunately, more and more sour crude oils are being produced worldwide. Fortunately, not all sulfur bearing compounds in crude oil are corrosively active. So, the information that crude oil operators need is not the level of total sulfur in the sample, but rather they need to know the level of reactive sulfur in the sample.

Current methods of for determining reactive sulfur content tend to be complicated and time consuming. For example, one technique is to take a sample of the crude oil or intermediate stream and perform direct catalytic conversion of the non-thiophenic sulfur to mercaptans and hydrogen sulfide. This is done with an alumina catalyst at 450° C. Then, the mercaptans concentration is determined by titration and the hydrogen sulfide is measure by gas chromatography. One shortcut refiners are inclined to use is simply to measure the total sulfur content and blend down total sulfur content with sweeter crude oils or sweeter hydrocarbon intermediate streams. This is done with simple X-ray Florescence (XRF) which can be done. However, while this short cut will reduce corrosion risk with respect to organosulfur compounds, the drive to minimize expense in the production of fuels and refined products means using less sweet components which are more expensive than sour components.

What is desired is a fast and simple process to determine with reasonable accuracy the content of reactive sulfur compounds.

### BRIEF SUMMARY OF THE DISCLOSURE

The invention more particularly relates to a process for blending source refinery streams to create a resultant refinery stream that reasonably optimizes feedstock costs while also balancing corrosion risk in refinery metallurgy presented by reactive sulfur in the source refinery streams. The process includes providing a fluid sample of a source refinery stream containing reactive sulfur and measuring the total sulfur content of the sample of the source refinery stream. Iron powder is provided with a surface area of at least about 0.01 m2/g and the iron powder is combined with the fluid sample for a period of time and at a selected temperature of at least approximately the temperature to which the resultant refinery stream may be subjected within the refinery so as to cause the reactive sulfur to react with iron to form solid ferric sulfide (FeS) thereby gathering a substantial portion of the reactive sulfur from the fluid sample into a solid phase. The solid phase is separated from the fluid creating a lower corrosion risk fluid sample and the total sulfur is measured in the lower corrosion risk fluid sample to thereby determine by subtraction the portion of the original total sulfur content of the fluid sample containing reactive sulfur and the source refinery stream is blended with another source refinery stream to create a desired resultant blend for subsequent processing in the refinery.

### BRIEF DESCRIPTION OF THE DRAWINGS

A more complete understanding of the present invention and benefits thereof may be acquired by referring to the following description taken in conjunction with the accompanying drawings in which:

The FIGURE is a chart showing sulfur concentrated in the heavier boiling point fractions of crude oil and what fractions of the sulfur content comprise reactive sulfur and thiophenic sulfur where the real concern to a refinery operator is the reactive sulfur of a hydrocarbon stream.

### DETAILED DESCRIPTION

Turning now to the detailed description of the preferred arrangement or arrangements of the present invention, it should be understood that the inventive features and concepts may be manifested in other arrangements and that the scope of the invention is not limited to the embodiments described or illustrated. The scope of the invention is intended only to be limited by the scope of the claims that follow.

The invention relates to a method for determining corrosiveness of sulfur in a hydrocarbon stream prior to subjecting that stream to a refining process for which corrosion of the metallurgy of the refining equipment in that process is a concern. In accordance with the present invention, examples of typical refinery streams which may be tested include but are not limited to atmospheric distillates of crude oil, vacuum distillates of crude oil and the like. Other hydrocarbon streams which can be tested using the method of the present invention include a wide variety of crude oils, heavy oils, light oils and the like. The method of the present invention advantageously allows for measuring the corrosiveness caused by reactive sulfur in the fluid. Referring to the FIGURE, detailed analysis of the sulfur content of a crude oil where the sulfur is not evenly distributed across all boiling point fractions. Sulfur tends to concentrate in the heavier oils that have the higher boiling points. One additional point from the FIGURE is that the concern for refinery operations is not the total sulfur content but turns out to be the reactive sulfur content of the stream.

Certain thiophenes contain sulfur, but do not react with refinery metallurgy at the conditions to which the fluid will be subjected. As such, those organosulfur compounds are typically not a concern. But elemental sulfur is a concern. Mercaptans, aliphatic sulfurs and hydrogen sulfide are concerns. While some refinery equipment may be provided with sulfur resistant metallurgy, most is not. Sulfur caused corrosion is basically a reaction that causes the iron in the steel equipment to combine and form ferric sulfide (FeS).

The measurement process of the present invention simply replicates the chemistry of the corrosion process using high surface-area iron powder that is preferably blended and mixed with a sample of the hydrocarbon material to accelerate the reaction and convert as much of the reactive sulfur as practical. Since a total sulfur measurement is relatively easy to obtain using x-ray florescence, an initial measurement of total sulfur is taken with a follow-up measurement to be taken after the reactive sulfur is bound up with iron powder. After the initial sulfur measurement is taken, the sample is combined with a sufficient quantity of iron powder to capture to react with all of the reactive sulfur available in the sample. Clearly, the available iron powder for this measurement should not be a limiting factor. The reactive sulfur reacts at temperature to form FeS so that the before and after sulfur measurements effectively provide a reasonable indication of the total content of the reactive sulfur in the larger amount of the hydrocarbon material. This is done with time, temperature, surface area and agitation and any other techniques for driving the corrosion reaction forward. The reasonable amount of time is preferably about an hour, but reasonable testing with various hydrocarbon samples and iron powders may provide a more optimal amount of time. The optimal temperature is believed to closely approximate the highest temperature in the prospective refining process. Higher temperatures may cause otherwise unreactive organosulfurs to become reactive and may lead to an elevated measurement of the reactive sulfur. Since one aspect of optimizing financial performance of a refinery is to run lower cost, but more corrosive feedstocks, any misinformation as to the reactive sulfur or the risk to the refinery metallurgy may cause suboptimal blending of the hydrocarbon material and opportunity loss. Higher surface area of the iron powder makes for more opportunities for the corrosion reaction and avoids the need to weight any coupons. The FeS is separated from the liquid along with any excess iron powder effectively removing the reactive sulfur from the sample. So, excess iron powder is supplied to the sample or obtaining finer powder may be obtained for the measurement to assure that as much reactive sulfur is converted to FeS. This again is subject to testing and experience balanced by the cost of lower and higher surface area iron powder samples.

In accordance with the present invention, a sample of fluid to be tested is obtained, for example from a refinery stream or other hydrocarbon stream, or as a fixed sample or the like. The sample is mixed with the powder or particulate iron that preferably has zero valence, sometimes indicated as Fe⁰ having a high surface area, preferably under an inert atmosphere and at subjected to projected temperatures or conditions of the planned refinery process to be evaluated for corrosiveness.

The solid non-reacted iron and the iron sulfide is then removed through filtering from the resulting mixed product. The unreacted sulfur concentration in the remaining organic phase is then measured using well known and conventional methods such as the x-ray florescence described above.

In accordance with the preferred embodiment of the present invention, the iron powder to be mixed is preferably a high surface area iron powder, preferably having a surface area of at least about 0.01 m2/g, more preferably between about 0.05 and 2 m2/g and most preferably between about 0.1 and about 1 m2/g. In addition, the powder preferably has an average particle size of less than or equal to about 50 μm.

The iron powder is preferably mixed with the stream or sample to be evaluated in amounts sufficient to provide a molar ratio of iron to sulfur in the stream of at least about 1:2, and preferably greater than about 80:1. In accordance with the present invention, it has been found that this step advantageously provides for the content of the sulfur to be the limiting factor in the reactions which take place, thereby providing an accurate and reliable measurement of the reactive sulfur content.

Corrosiveness can vary with temperature, and it is therefore preferred to carry out the contacting or mixing step at a known temperature, preferably at a range of known temperatures, whereby the measure of corrosiveness is correlated to corrosiveness at the particular temperature. In this manner, a range of corrosiveness values can be provided for the range of temperatures.

As set forth above, the contacting or mixing step is preferably carried under an inert atmosphere such as nitrogen or argon, for example. This atmosphere is inert with respect to the iron powder so as to advantageously avoid oxidation of same. Of course, other types of iron inert atmospheres could be used.

As set forth above, it should be appreciated that the method of the present invention provides an indirect measure of the reactive sulfur content. Further, the method of the present invention is particularly advantageous for use in testing a variety of hydrocarbon fluids found in a refinery.

It should also be appreciated that the method of the present invention is carried out using simple particulate or powdered iron, which is readily available and therefore contributes to the economic value of the present invention. Finally, the method provides for measurements with a very high degree of accuracy and repeatability, which can be carried out in virtually any desired location.

One of the powerful aspects of the present invention is that it does not have to react all the sulfur to provide the information needed by a refinery operator. It is a conditional test that can answer the basic question, such as: “How much sulfur is reactive at 475° F?” If 475° F. is the highest temperature that the stream will experience, one does not need to know the answer to the question: “How much sulfur will react at 550° F?” And it should be noted that the amounts of reactive sulfur will likely differ at the two different temperatures if there are a variety of sulfur species in the fluid. The test procedure is not intended to determine total sulfur or the amount of reactive sulfur at ultra-high temperature conditions. That would be superfluous information to one running a refinery.

It should also be understood that while most corrosion concerns in a refinery are for carbon steel equipment, there may be concerns about other steel alloys. In following the teachings of the present invention, powder of various metallurgical compositions may be tested to provide a measure of the portion of the total sulfur that is reactive to that specific metallurgy at the test temperature. For instance, if one were concerned about a certain chromium or molybdenum steel, a powder comprising the alloy could be used where the before and after measurements of total sulfur of the sample indicates the content of the sample that creates a corrosion risk to the metallurgy of the refinery units.

As an example of the measurements of total reactive sulfur, the test procedure used samples of four crude blends is shown in Table 1 below. The total sulfur content in weight percent is shown along with the measured reactive sulfur in weight percent. The reactive sulfur is important for understanding the corrosion risk to refinery equipment.

TABLE 1

| | | Difference in wt % Sulfur |
|---|---|---|
| Crude | Total wt % Sulfur | in Sample after iron powder |
| Blend | in Feed, ppm | treatment at 260° C. |
| A | 2.74 | 1.36 |
| B | 2.92 | 1.43 |
| C | 3.46 | 1.57 |
| D | 3.56 | 1.66 |

In closing, it should be noted that the discussion of any reference is not an admission that it is prior art to the present invention, especially any reference that may have a publication date after the priority date of this application. At the same time, each and every claim below is hereby incorporated into this detailed description or specification as additional embodiments of the present invention.

Although the systems and processes described herein have been described in detail, it should be understood that various changes, substitutions, and alterations can be made without departing from the spirit and scope of the invention as defined by the following claims. Those skilled in the art may be able to study the preferred embodiments and identify other ways to practice the invention that are not exactly as described herein. It is the intent of the inventors that variations and equivalents of the invention are within the scope of the claims while the description, abstract and drawings are not to be used to limit the scope of the invention. The invention is specifically intended to be as broad as the claims below and their equivalents.

1. A process for blending source refinery streams to create a resultant refinery stream that reasonably optimizes feedstock costs while also balancing corrosion risk in refinery metallurgy presented by reactive sulfur in the source refinery streams, wherein the process comprises:
providing a fluid sample of a source refinery stream containing reactive sulfur;
measuring the total sulfur content of the sample of the source refinery stream;
providing iron powder having a surface area of at least about 0.01 m²/g;
combining the iron powder with the fluid sample for a period of time and at a selected temperature of at least approximately the temperature to which the resultant refinery stream may be subjected within the refinery so as to cause the reactive sulfur to react with iron to form solid ferric sulfide (FeS) thereby gathering a substantial portion of the reactive sulfur from the fluid sample into a solid phase;
separating the solid phase from the fluid creating a lower corrosion risk fluid sample;
measuring the total sulfur in the lower corrosion risk fluid sample to thereby determine by subtraction the portion of the original total sulfur content of the fluid sample containing reactive sulfur; and
blending the source refinery stream with another source refinery stream to create a desired resultant blend for subsequent processing in the refinery.

2. The process according to claim 1, wherein said iron powder has an average particle size of less than or equal to about 50 μm.

3. The process according to claim 1, wherein the step for combining provides the iron powder in the fluid sample at the selected temperature for at least 45 minutes.

4. The process according to claim 1, wherein the step for combining provides the iron powder in the fluid sample at the selected temperature for at least 60 minutes.

5. The process according to claim 1, wherein said step of separating the solid phase comprises filtering the solid phase from the fluid.

6. The process according to claim 1, wherein said combining the iron powder with the sample of the fluid further comprises mixing the iron powder and the fluid sample so as to provide a substantially homogeneous mixture of the powder and the fluid sample.

7. The process according to claim 1, wherein said combining step is carried out under an inert atmosphere.

8. The process according to claim 1, wherein said refinery stream is selected from the group consisting of atmospheric distillates, vacuum distillates and mixtures thereof.

9. The process according to claim 1, wherein said fluid is a crude oil.

10. The process according to claim 1, wherein said fluid is a boiling point fraction of crude oil where at least 50% of the fraction has a boiling point in excess of 500° F.

11. The process according to claim 1, wherein said iron powder has a surface area of between about 0.05 and about 2 m2/g.

12. The process according to claim 1, wherein said powder is present in an amount sufficient to react all of the sulfur in the fluid.

13. The process according to claim 1 wherein said powder is present in an amount sufficient to react all of the reactive sulfur in the fluid.
